(19) United States
(12) Patent Application Publication
(10) Pub. No.: US 2010/0145314 A1
(43) Pub. Date: Jun. 10, 2010

(54) Catheter Cap Guard: A Catheter Accessory

(75) Inventor: Haim Hazan, Flushing, NY (US)

Correspondence Address: MCCARTER & ENGLISH, LLP STAMFORD, CANTERBURY GREEN, 201 BROAD STREET, 9TH FLOOR, STAMFORD, CT, 06901, US

(73) Assignee: Hoard Fantastic Corporation, Auburndale, NY (US)

(21) Appl. No.: 12/631,202

(22) Filed: Dec. 4, 2009

Related U.S. Application Data
(60) Provisional application No. 61/200,831, filed on Dec. 4, 2008.

(51) Int. Cl. A61M 25/00

(52) U.S. Cl. 604/544

(57) ABSTRACT

A cap guard for a urinary catheter is presented which extends the catheter away from the urinary meatus, in-line with the urethra, for a given distance thereby preventing/mitigating irritation to the penile head and urinary meatus during catheterization. Thus, the cap guard generally includes/defines a conduit extending away from the urinary meatus, in-line with the urethra. The conduit is typically configured to direct a catheter therethrough away from the head of the penis while the catheter is maintained in-line with the urethra. Thus, the conduit acts as a buffer between the penis and a bend/elbow region of the catheter. Systems and methods utilizing the cap guard architecture are further provided. Systems generally include the cap guard and a catheter. Methods generally involve steps of providing a cap guard and securing the cap guard relative to the penis. Methods are also provided including steps of inserting a catheter into a patient's penis and either creating buffer zone between the penis and a bend in the catheter or maintaining the catheter in-line with the urethra for some distance extending from the penis.

### CROSS REFERENCE TO RELATED APPLICATIONS

The present application claims the benefit of a co-pending, provisional patent application filed on Dec. 4, 2009, and assigned Ser. No. 61/200,831. The entire content of each of the foregoing provisional patent application are incorporated herein by reference.

### BACKGROUND

1. Technical Field

The present disclosure relates to apparatus, systems and methods for urinary catheterization.

2. Background Art

In urinary catheterization, a plastic tube known as a urinary catheter is inserted through a patient's urethra into the patient's bladder. Catheterization may be used to drain urine from the patient's bladder, e.g., for collection in a drainage bag. Catheterization may also be used to inject fluids into a patient's bladder for diagnosis/treatment purposes (e.g., an irrigation catheter has a separate lumen to carry irrigation fluid into the bladder). Catheterization is useful in many situations including, in particular, when dealing with anesthetized, sedated or comatose patients, patients with paralysis or injury, patients with incontinence, patients with enlarged prostates, e.g., due to Benign Prostatic Hyperplasia (BPH), prostate cancer, etc., and patients with acute urinary retention.

One popular basic urinary catheter design is a Foley catheter. The Foley catheter is retained within the bladder by a balloon at the tip which, after insertion, may be inflated, e.g., with sterile water. A second basic type of urinary catheter is the Robinson catheter. Contrasted with Foley catheter, the Robinson catheter does not include a balloon and therefore cannot remain in place independently. Thus, the Robinson catheter is typically used for short term drainage, whereas the Foley catheter is utilized as more of a long term fixture.

Catheterization for male patients typically involves inserting the catheter into the urinary tract through the cavernous portion of the urethra (also known as the spongy urethra or penile urethra) which runs along the length of the ventral surface of the penis. The catheter then passes through the membranous portion of the urethra (passed the external urinal sphincter) and through the prostatic and pre-prostatic portions of the urethra into the bladder. Ordinarily, the length of the cavernous portion of the urethra fluctuates. Thus, catheterization for a male patient must include some freedom of movement to account for such fluctuations. For example, catheterization for a male patient using a Foley catheter must account for pistoning of the catheter at the urinary meatus (the external urethral orifice).

Figures A, B and C are schematics depicting a conventional catheter arrangement 5. In the depicted catheter arrangement 5, a catheter 10 is inserted into the bladder through the urethra 22 via the urinary meatus 24 of the penis 20. As may be observed by comparing Figures A and B, the catheter position relative to penis 20 may fluctuate due to a pistoning effect of the catheter 10. As discussed above, one of the primary reasons for inserting a catheter is to drain urine from the patient's bladder. Thus, referring specifically to Figures B and C, the catheter 10 may be connected to a drainage bag 30, e.g., for storing drained fluids. For mobile patients, the drainage bag 30 may be advantageously mounted relative to the patient 50, e.g., via a thigh strap 40. Thus, the catheter 10 may often include a bend/elbow-region 15 for directing the catheter 10 to the drainage bag 30. As best depicted in Figure B, the bend/elbow region 15 of the conventional catheter arrangement 5 may repeatedly contact/rub against the urinary meatus 24 of the penis 20, e.g., on account of the pistoning of the catheter 10. Thus, the conventional catheter arrangement 5 often results in irritation to the skin surrounding the urinary meatus 24. Such irritation may occur within 12-36 hours of insertion of the catheter 10 and may result in infection and/or more serious complications.

Thus, despite efforts to date, there remains a need for catheter arrangements which address/overcome the disadvantages of conventional catheterization, i.e., such as skin irritation caused by the conventional catheter arrangement in Figures A, B and C. These and other needs are addressed by the apparatus, systems and methods of the present disclosure.

### SUMMARY

Advantageous apparatus, systems and methods are provided herein for facilitating urinary catheterization. Specifically, a cap guard for a urinary catheter is presented which advantageously extends the catheter away from the urinary meatus, in-line with the urethra, e.g., before the catheter is directed to a drainage bag, thereby preventing/mitigating irritation to the penile head and urinary meatus during and after catheterization. Thus, the cap guard may include/define a conduit extending away from the urinary meatus, in-line with the urethra. The conduit may be advantageously configured to direct a catheter therethrough away from the head of the penis while the catheter is maintained in-line with the urethra (i.e., in a “neutral” position). Thus, the conduit may act as a buffer between the penis and a bend/elbow region of the catheter.

The cap guard may further include a means of attachment relative to the penis. Thus, the cap guard may include a mounting structure, e.g., a dome structure, configured to fit over the glans penis and secure relative thereto, e.g., wherein the conduit may be affixed relative to the apex of the dome structure. In exemplary embodiments, the dome structure may include a cylindrical base region as well as a plurality of curved struts extending from the base region and meeting at the apex of the dome. Thus, the mounting structure may advantageously include an “open air” design to allow for tissue aeration.

In exemplary embodiments, the cap guard may be advantageously divided into two or more sections, e.g., two halves, to facilitate placement around the penis, as well as to allow for placement after insertion of the catheter. Thus, e.g., the cap guard may include one more hinged regions, e.g., for hingedly associating the two halves of the cap guard, as well as one or more locking mechanisms, e.g., for securing the two halves of the cap guard in a closed position around the glans penis.

In exemplary embodiments, the cap guard may include an interchangeable band for sizing the cap guard, e.g., relative to the penis. Thus, the cap guard may define a channel relative to an inner surface of the base of the mounting structure, e.g., the base region of the dome structure, for receiving the interchangeable band. The band may be may include a gel material or other spongy material to provide added comfort for the patient.

In further exemplary embodiments, a bandage may be secured relative to the base of the glans penis for association with the cap guard. The bandage may provide protection for the skin around the base of the glans penis as well as facilitate mounting/securing the cap guard relative thereto. The bandage may also facilitate sizing of the cap guard relative to the penis. In exemplary embodiments, the bandage may include a Velcro material on the exterior surface and a sterile gauze material on the interior surface. The Velcro material may be advantageously used to secure the bandage relative to the penis and/or to secure the cap guard relative to the bandage.

Systems and methods utilizing the cap guard architecture are further provided according to the present disclosure. Systems utilizing the cap guard architecture generally include the cap guard and a catheter. Similarly, methods utilizing the cap guard architecture generally include steps of:
providing a cap guard, as described above and
securing the cap guard relative to a patient's penis (e.g., such that the catheter is directed through the conduit and away from the penis whereby the catheter is maintained in-line with the urethra).

Advantageously, the cap guard may be secured relative to the penis after insertion of the catheter.

Methods are also provided including steps of inserting a catheter into a patient's penis and either (i) creating buffer zone between the penis and a bend in the catheter or (ii) maintaining the catheter in-line with the urethra for some distance extending from the penis.

Additional features, functions and benefits of the disclosed apparatus, systems and methods will be apparent from the description which follows, particularly when read in conjunction with the appended figures.

### BRIEF DESCRIPTION OF THE DRAWINGS

To assist those of ordinary skill in the art in making and using the disclosed systems and methods, reference is made to the appended figures, wherein:

Figures A, B and C depict a conventional prior art catheter arrangement, i.e., without a cap guard.

FIGS. 1 and 2 depict an exemplary cap guard according to the present disclosure, in closed and open positions, respectively.

FIG. 3 depicts interchangeable bands for sizing the cap guard of FIG. 1.

FIGS. 4 and 5 depict placing and securing the cap guard of FIG. 1 relative to a patient.

FIG. 6 depicts a bandage for association with the cap guard of FIG. 1.

### DESCRIPTION OF EXEMPLARY EMBODIMENT(S)

Advantageous apparatus, systems and methods are provided according to the present disclosure for facilitating urinary catheterization. In particular, the present apparatus systems and methods prevent/mitigate irritation to the penile head and urinary meatus during catheterization by creating a buffer region between the head of the penis and a bend/elbow region of the catheter.

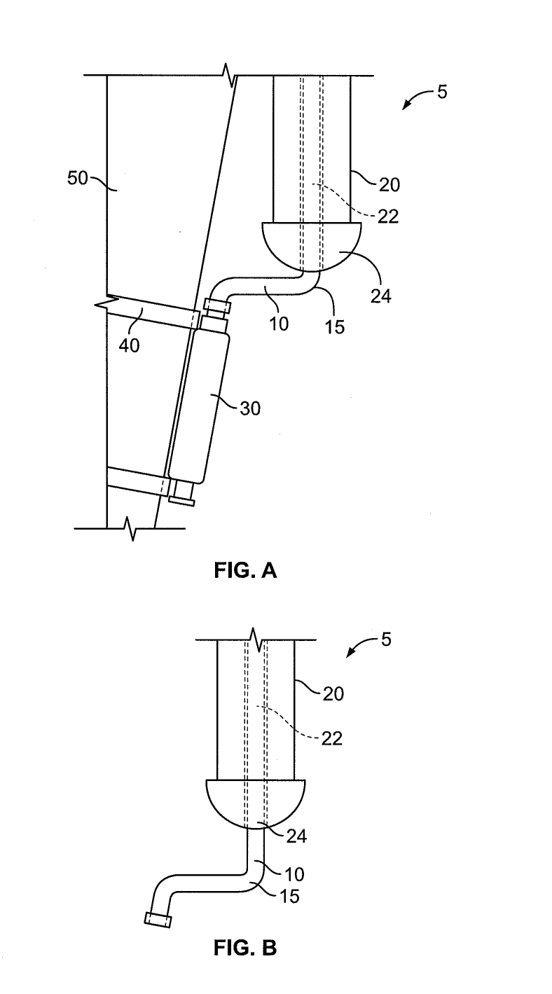

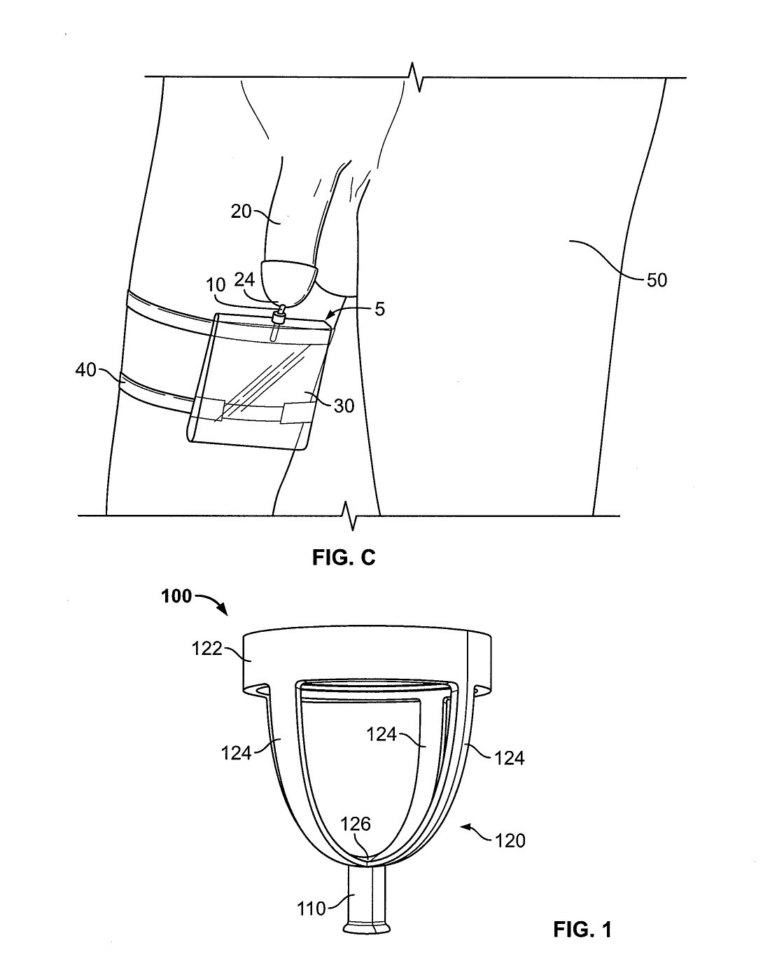

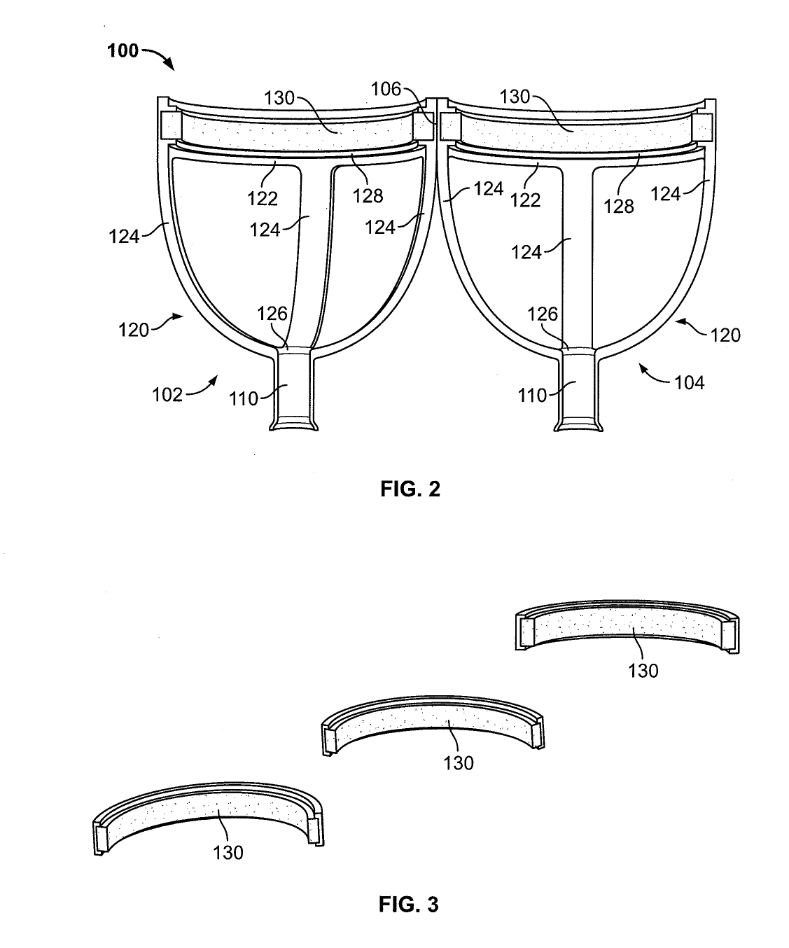

With initial reference to FIGS. 1 and 2, an exemplary cap guard 100 for a urinary catheter is depicted. The cap guard 100 is configured to advantageously enable extending a catheter away from the urinary meatus of a patient's penis, in-line with the urethra for a given distance, thereby preventing/mitigating irritation to the penile head and urinary meatus during and after catheterization. Thus, the cap guard 100 may include/define a conduit 110 adapted to receive a catheter therethrough. The cap guard 110 may further include/define a dome structure 120 configured to fit over the glans penis and secure relative thereto, e.g., wherein the conduit 110 is affixed relative to the apex 126 of the dome structure 120. In exemplary embodiments, the dome structure 120 may include a cylindrical base region 122 and a plurality of curved struts 124 extending from the base region 122 and meeting at the apex 126 of the dome. Thus, the cap guard 100 may advantageously include an “open air” design to allow for tissue aeration. The cap guard 110 construction may further allow for flexibility. In preferred embodiments, the cap guard is constructed as a single integral unit using a suitable polymer/plastic.

As best depicted in FIG. 2, the cap guard 100 may advantageously be divided into two or more sections, e.g., halves 102 and 104, to facilitate placement around the glans penis, as well as to allow for placement after insertion of the catheter. Thus, e.g., the cap guard may include one more hinged regions 106, e.g., for hingedly associating the halves 102 and 104 of the cap guard 100, as well as one or more locking mechanisms (not depicted), e.g., for securing the halves 102 and 104 of the cap guard 100 in a closed position, such as depicted in FIG. 1. The locking mechanism may include any known locking means, e.g., a latch, a buckle, a strap, a snap, etc.

Referring now to FIGS. 2 and 3, the cap guard 100 may include one or more interchangeable bands 130 for sizing the cap guard, e.g., relative to the penis. Thus, the cap guard may define a channel 128 relative to the base region 122 of the dome structure 120 for receiving an interchangeable band 130. The interchangeable band 130 may include a gel material or other spongy material to provide added comfort for the patient.

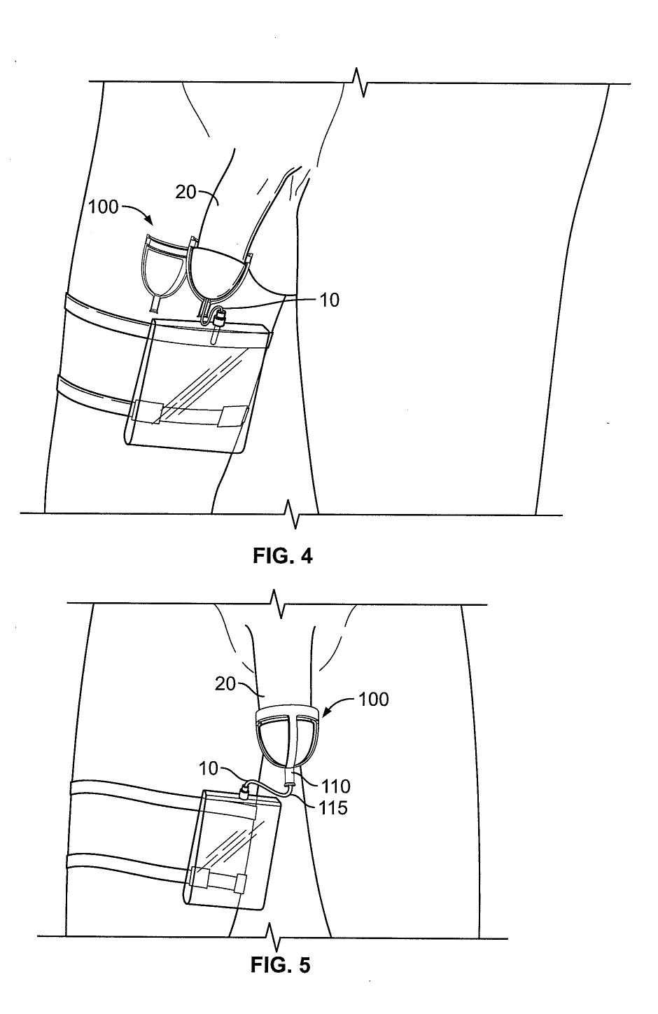

Referring now to FIGS. 4 and 5, exemplary steps of placing and securing the cap guard 100 relative to the penis 20 are depicted, respectively. Specifically, the cap guard 100 may be fitted around both an inserted catheter 10 and the penis 20 while in an open position, as depicted in FIG. 4, and then secured in a closed position as depicted in FIG. 5. As illustrated, the conduit 110 advantageously receives and directs the catheter 10 away from the head of the penis 20 while maintaining the catheter 10 in-line with the urethra (i.e., in a “neutral” position). Typically, e.g., for a Foley catheter, the diameter of the conduit 110 may be slightly larger than the diameter of the catheter 10 (to allow for freedom of motion of the catheter 10). Alternatively, e.g., for a Robinson catheter, the diameter of the conduit 110 may be slightly smaller than the diameter of the catheter 10 (to assist in holding the catheter 10 in place). In exemplary embodiments, the conduit 110 extends the catheter 10 in the neutral position for approximately 0.50-0.75 inches. Thus, the conduit 110 may act as a buffer between the penis 20 and a bend/elbow region 15 of the catheter 10.

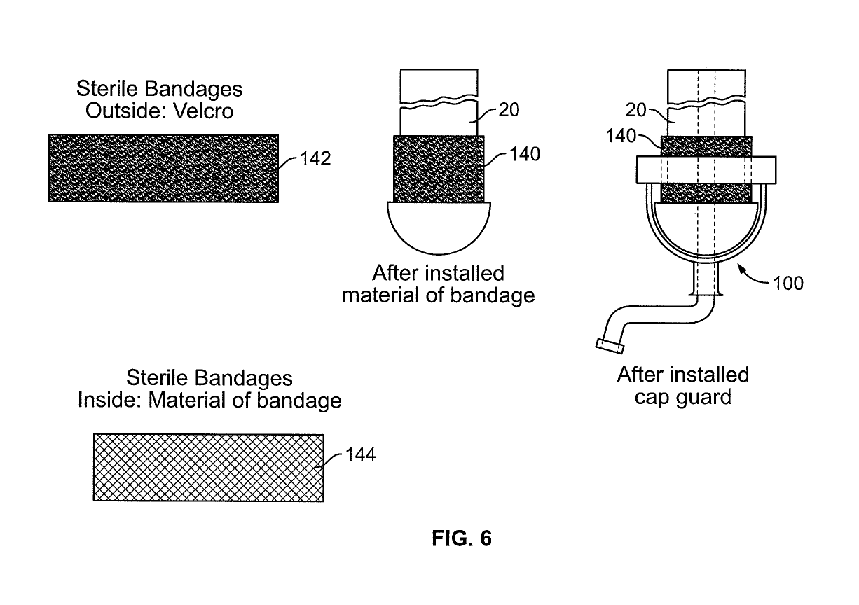

Referring now to FIG. 6, a bandage 140 may secured relative to the base of the glans penis for association with the cap guard 100. The bandage 140 may provide protection for the skin around the base of the glans penis as well as facilitate mounting/securing the cap guard 100 relative thereto. The bandage may also facilitate sizing of the cap guard relative to the penis 20. In exemplary embodiments, the bandage 140 may include a Velcro material 142 on the exterior surface and a sterile gauze material 144 on the interior surface. The Velcro material may be advantageously used to secure the bandage relative to the penis 20 and/or to secure the cap guard 100 relative to the bandage 140.

Although the present disclosure has been described with reference to exemplary embodiments and implementations thereof, the disclosed apparatus, systems and methods are not limited to such exemplary embodiments/implementations. Rather, as will be readily apparent to persons skilled in the art from the description provided herein, the disclosed apparatus, systems and methods are susceptible to modifications, alterations and enhancements without departing from the spirit or scope of the present disclosure. Accordingly, the present disclosure expressly encompasses all such modifications, alterations and enhancements within the scope hereof.

What is claimed:

1. A cap guard for a urinary catheter, the cap guard comprising:
a. a mounting structure configured to secure relative to a patient's penis and
b. a conduit fixed relative to the mounting structure and configured such that, when the mounting structure is secured relative to the penis, the conduit extends a predetermined distance away from the penis and in-line with the urethra, wherein the conduit is further configured to receive a catheter therethrough and thereby maintain the catheter in-line with the urethra for the predetermined distance.

2. The cap guard of claim 1, wherein the conduit is adapted to act as a buffer between the penis and a bend in the catheter.

3. The cap guard of claim 1, wherein the mounting structure is a dome structure configured to fit over the glans penis and secure relative thereto, wherein the conduit is fixed relative to the apex of the dome structure.

4. The cap guard of claim 3, wherein the dome structure includes a cylindrical base region and a plurality of curved struts extending from the base region and meeting at the apex of the dome structure.

5. The cap guard of claim 1, wherein the mounting structure includes an open air design to allow for tissue aeration.

6. The cap guard of claim 1, wherein the cap guard is configured such that it may be mounted relative to the penis after insertion of the catheter into the penis.

7. The cap guard of claim 1, wherein the cap guard is divided into two or more sections to facilitate placement around the penis.

8. The cap guard of claim 7, further comprising one or more hinged regions for hingedly associating two or more of the two or more sections such that the cap guard may be switched between open and closed positions.

9. The cap guard of claim 7, further comprising one or more locking mechanisms for securing the cap guard in a closed position.

10. The cap guard of claim 1, wherein the mounting structure is adapted to cooperate with an interchangeable band for sizing relative to the penis.

11. The cap guard of claim 10, wherein an inner surface of the base of the mounting structure defines a channel for receiving the interchangeable band.

12. The cap guard of claim 1, wherein the cap guard is associated with a bandage secured relative to the base of the glans penis, wherein the bandage at least one of: (i) provides protection for the skin around the base of the glans penis during mounting of the cap guard relative thereto and (ii) facilitates securing the mounting structure relative to the penis.

13. The cap guard of claim 12, wherein the bandage includes a Velcro material on the exterior surface and a sterile gauze material on the interior surface, wherein the Velcro material at least one of: (i) facilitates securing the bandage relative to the penis and (ii) facilitates securing the mounting structure relative to the penis.

14. The cap guard of claim 1, wherein the predetermined distance is about 0.5-0.75″.

15. A catheterization system comprising:
a. a catheter and
b. a cap guard including
i. a mounting structure configured to secure relative to a patient's penis and
ii. a conduit fixed relative to the mounting structure and configured such that, when the mounting structure is secured relative to the penis, the conduit extends for a predetermined distance away from the penis in-line with the urethra, wherein the conduit is further configured to receive the catheter therethrough and thereby maintain the catheter in-line with the urethra for some distance.

16. The catheterization system of claim 15, wherein the conduit is slightly larger in diameter than the catheter thereby allowing for some freedom of motion.

17. The catheterization system of claim 15, wherein the conduit is slightly smaller in diameter than the catheter thereby facilitating holding the catheter in place.

18. A catheterization method comprising:
a. providing a catheter cap guard including a mounting structure and a conduit fixed relative to the mounting structure
b. securing the mounting structure relative to a patient's penis such that the conduit extends for a predetermined distance away from the penis and in-line with the urethra, wherein the conduit is further configured to receive a catheter therethrough and thereby maintain the catheter in-line with the urethra for some distance.

19. The method of claim 18, further comprising an initial step of inserting the catheter into the penis, wherein the securing the mounting structure includes fitting the catheter cap over both the penis and the catheter.

20. A catheterization method comprising:
a. inserting a catheter into a patient's penis
b. creating buffer zone between the penis and a bend in the catheter.

21. A catheterization method comprising:
a. inserting a catheter into a patient's penis
b. maintaining the catheter in-line with the urethra for a predetermined distance extending from the penis.
